SEQUENCE LISTING (24 sequences)

SEQ ID NO: 1 - DNA, 19 bp, synthetic construct
  source 1..19: mol_type = other DNA
caagttccccggcgatgtc

SEQ ID NO: 2 - DNA, 21 bp, synthetic construct
  source 1..21: mol_type = other DNA
ttctggtcaaggtcacctgtc

SEQ ID NO: 3 - DNA, 23 bp, synthetic construct
  source 1..23: mol_type = other DNA
acatggagactttgtcccttttg

SEQ ID NO: 4 - DNA, 21 bp, synthetic construct
  source 1..21: mol_type = other DNA
ttggctgagtggtagagtccc

SEQ ID NO: 5 - DNA, 21 bp, synthetic construct
  source 1..21: mol_type = other DNA
ctggacagccagacactaaag

SEQ ID NO: 6 - DNA, 20 bp, synthetic construct
  source 1..20: mol_type = other DNA
ctcgcggcaagtcttcagag

SEQ ID NO: 7 - DNA, 20 bp, synthetic construct
  source 1..20: mol_type = other DNA
tcagagccaaagcagtgagc

SEQ ID NO: 8 - DNA, 23 bp, synthetic construct
  source 1..23: mol_type = other DNA
gccgtgtagataaactcgatgtc

SEQ ID NO: 9 - DNA, 21 bp, synthetic construct
  source 1..21: mol_type = other DNA
aacagcttggtggtgatcgtc

SEQ ID NO: 10 - DNA, 21 bp, synthetic construct
  source 1..21: mol_type = other DNA
catagcggtatagacagccca

SEQ ID NO: 11 - DNA, 20 bp, synthetic construct
  source 1..20: mol_type = other DNA
aactggcaccaatgagtccg

SEQ ID NO: 12 - DNA, 23 bp, synthetic construct
  source 1..23: mol_type = other DNA
ccaaaaggagtaagtcagccaag

SEQ ID NO: 13 - DNA, 22 bp, synthetic construct
  source 1..22: mol_type = other DNA
agaaatcccttcacggtctaca

SEQ ID NO: 14 - DNA, 23 bp, synthetic construct
  source 1..23: mol_type = other DNA
gtcaccgataatgtcacgattgt

SEQ ID NO: 15 - DNA, 20 bp, synthetic construct
  source 1..20: mol_type = other DNA
aggttgggctactccaggac

SEQ ID NO: 16 - DNA, 21 bp, synthetic construct
  source 1..21: mol_type = other DNA
ggtgagttgttgtctggcttc

SEQ ID NO: 17 - DNA, 21 bp, synthetic construct
  source 1..21: mol_type = other DNA
tccagactccgatcatcaagc

SEQ ID NO: 18 - DNA, 23 bp, synthetic construct
  source 1..23: mol_type = other DNA
gctcatggtgttcagaattgtgt

SEQ ID NO: 19 - DNA, 19 bp, synthetic construct
  source 1..19: mol_type = other DNA
ctcccgtggcttctagtgc

SEQ ID NO: 20 - DNA, 22 bp, synthetic construct
  source 1..22: mol_type = other DNA
gccttagtttggacaggatctg

SEQ ID NO: 21 - DNA, 20 bp, synthetic construct
  source 1..20: mol_type = other DNA
ggctctattcccctccatcg

SEQ ID NO: 22 - DNA, 22 bp, synthetic construct
  source 1..22: mol_type = other DNA
ccagttggtaacaatgccatgt

SEQ ID NO: 23 - DNA, 19 bp, synthetic construct
  source 1..19: mol_type = other DNA
cttcgacgtgacagacgct

SEQ ID NO: 24 - DNA, 22 bp, synthetic construct
  source 1..22: mol_type = other DNA
gcaggggcagtgtaaacttatt

(19) United States
(12) Patent Application Publication
(10) Pub. No.: US 2023/0194550 A1
(43) Pub. Date: Jun. 22, 2023

(54) STIMULATION OF THE HEALING PROCESS ON THE RETINAL PIGMENT EPITHELIUM AFTER R:GEN WITH RTF TECHNOLOGY

(71) Applicant: Lutronic Vision, Inc., Bellerica, MA (US)

(72) Inventors: Stan G. LOUIE, Fullerton, CA (US); Mark S. HUMAYAN, Glendale, CA (US); Juan Carlos MARTINEZ-CAMARILLO, Los Angeles, CA (US); Jhung Won VOJIR, Huntingdon Valley, PA (US)

(73) Assignee: Lutronic Vision, Inc., Bellerica, MA (US)

(21) Appl. No.: 17/898,757

(22) Filed: Aug. 30, 2022

Related U.S. Application Data
(63) Continuation of application No. PCT/US2021/031314, filed on May 7, 2021.
(60) Provisional application No. 63/021,507, filed on May 7, 2020.

(51) Int. Cl. G01N 33/68; C12Q 1/6883; A61N 5/067

(52) U.S. Cl. CPC G01N 33/6893; C12Q 1/6883; A61N 5/067; C12Q 2600/158; G01N 2800/164; G01N 2800/52; A61F 2009/00863

(57) ABSTRACT

The effect of laser stimulation, e.g., R:GEN, of the RPE and its impact on MMPs and RAAS pathways are used to guide patient therapies. Certain biomarkers, namely MMPs, TIMPs, and components associated with RAAS, are effective indicators of healing response levels generated by the patients undergoing the therapy. An eye disease is diagnosed in a patient and a first biomarker sample is obtained from a biomatrix, e.g., patient's blood in containers with protease inhibitors. An initial subthreshold laser treatment is then performed on the eye. By monitoring the presence, amount, and relative levels of one or more of the above biomarkers as the patient heals, it is determined when the patient's body has sufficiently responded to the previous treatment, such that retreatment may be appropriate. The present disclosure demonstrates effective treatment of eye diseases, e.g., dry age-related macular degeneration, which utilize laser treatment alone or in combination with other treatments.

### CROSS REFERENCE TO RELATED APPLICATIONS

This application is a continuation of International Application No. PCT/US2021/031314, filed May 7, 2021, which claims the benefit of U.S. Provisional Patent Application No. 63/021,507, filed on May 7, 2020, which are incorporated by reference as if disclosed herein in their entireties.

### INCORPORATION OF SEQUENCE LISTING

The contents of the file named “Sequences_replacement.xml”, which was created on Dec. 20, 2022 and is 21.7 KB in size, are hereby incorporated by reference in their entireties.”

### BACKGROUND

Intense efforts have been ongoing to develop effective therapies for the treatment of dry age-related macular degeneration (“AMD”) and other retinal degenerative diseases. These efforts have included the development of photobiomodulation laser therapy capable of selectively stimulating Retinal Pigment Epithelial (“RPE”) cell. One exemplary system is the R:GEN® laser system developed by Lutronic that can preferentially stimulate melanosomes found specifically in RPE. The R:GEN system is a Q-switched frequency-double neodymium-doped yttrium lithium fluoride (Nd:YLF) laser-emitting 527 nm wavelength that specifically targets melanosomes in RPE. Using microsecond pulses over a larger period of exposure can stimulate RPE without damaging non-melanosome containing cells. Consequently, repeated doses of energy increase the intracellular temperature to develop a short-lived microbubble. Such a bubble increases the cell volume enough to disrupt the targeted RPE cell membrane. The R:GEN system uses two methods for the detection of microbubbles: 1) optoacoustic (OA) measurements detect pressure changes due to waves formed by microbubble formation, and 2) a reflectometry (RM) measurement detects microbubble formation through changes in light scattering in RPE. The use of both techniques results in Real Time Feedback (RTF) confirmation of a microbubble which will subsequently cease further energy delivery. With proper RTF values, treatment spots will be visible by fluorescein angiography (FA), which means selective RPE damage. Previous clinical studies have improved and validated the usage of the RTF technology.

Tissue response to either injury or infection will induce inflammation, thus recruiting immune cells to the affected site. Matrix metalloproteases (MMPs) are upregulated to degrade extracellular matrix (ECM) to provide cellular access. MMPs are secreted by the RPE and choroidal cells, as well as found in the Bruch's membrane. MMP2, MMP3 and MMP9 have been studied in relation to AMD. Moreover, MMP9 can increase RPE cell permeability by increasing expression of transforming growth factor beta (TGF-β), where its levels in plasma correlated with disease progression in AMD. MMP9 was also found to be involved in the wound healing process such as in acute lung injury, which is involved in extracellular matrix (ECM) remodeling. MMP1 and MMP8 are involved in infected chronic wound healing such as chronic venous ulcers, while MMP2 and MMP9 are involved in non-infected chronic wound healing. Once activated, MMPs can be modulated by tissue inhibitors of metalloproteases (TIMPs). MMP2 and TIMP-2 and -3 have been implicated in Müller glia reprogramming. Clear understanding what role MMPs play in the wound healing is still unclear, although transforming growth factor-beta (TGF-β) is thought to be involved in this process. TGF-β has been shown to promote tissue fibrosis.

The renin-angiotensin-aldosterone system (RAAS) is a regulator of hemodynamics and homeostasis such as blood pressure control. However, its importance in wound healing is still emerging. Key regulators of ocular pressure have been associated with elevated ocular levels of angiotensin II (Ang-II), which is produced locally. RAAS peptides are also upregulated in dermal tissue following radiation and thermal injuries. More importantly, acceleration in wound repair has been associated with administration of Ang-II metabolite, angiotensin (1-7), or Ang(1-7). Ang-II can itself promote rapid wound healing process which can also increase expression of TGF-β leading to fibrotic wound healing. In addition, Ang-II can also activate production of TIMP and pro-collagens in the fibroblast. In endothelial cells, Ang-II can regulate expression of MMP2 and MMP9. There are two receptors that can bind onto Ang II which are designated as angiotensin receptor 1 (AT1R) and 2 (AT2R). Ang-II binding onto AT1R can activate intracellular pathways leading to vasoconstriction and tissue fibrosis. In contrast, Ang-II binding onto AT2R activates promote non-fibrotic wound healing (see FIG. 9).

### SUMMARY

Aspects of the present disclosure are directed to a method for guiding treatment of a patient's eye including obtaining a first sample from a biomatrix from the patient, performing an initial treatment on the eye, measuring a concentration of one or more biomarkers in the first sample, obtaining one or more subsequent samples from a biomatrix of the patient after the initial treatment, measuring a concentration of the one or more biomarkers in the subsequent samples, and performing a subsequent treatment of the eye after the concentration of the one or more biomarkers returns to a predetermined threshold. In some embodiments, the one or more biomarkers include levels of one or more peptides, relative levels of the one or more peptides, ratio of these peptides, activity levels of the one or more peptides, relative activity levels of the one or more peptides, gene expression levels of one or more genes for the one or more peptides, relative expression levels of one or more genes for the one or more peptides, or combinations thereof. In some embodiments, the peptides include matrix metalloproteases (MMP), tissue inhibitors of metalloproteases (TIMPs), components of RAAS such as but not limited to Ang(1-9), AngII, Ang(1-7), Ang(1-10), MasR, AT1R, AT2R, angiotensin converting enzyme-1 (ACE1), ACE2, neprilysin (NEP), aminopeptidase isoforms, or combinations thereof. In some embodiments, the predetermined threshold is the concentration of the one or more biomarkers in the first sample. In some embodiments, the biomatrix includes the patient's blood, peripheral blood mononuclear cells, vitreous humor, aqueous humor, or combinations thereof. In some embodiments, the biomarker is measured in one or more of the patient's blood cellular components, serum, plasma, or combinations thereof. In some embodiments, the initial treatment and the subsequent treatment includes laser stimulation of the eye. In some embodiments, the laser is a subthreshold laser. In some embodiments, the laser is a frequency-double neodymium-doped yttrium lithium fluoride (Nd:YLF) laser.

In some embodiments, obtaining one or more subsequent samples from a biomatrix of the patient after the initial treatment occurs substantially immediately after the initial treatment. In some embodiments, obtaining one or more subsequent samples from a biomatrix of the patient after the initial treatment occurs at least one hour after the initial treatment. In some embodiments, the subsequent treatment occurs at least 7 days after the initial treatment.

Aspects of the present disclosure are directed to a method of identifying and quantifying one or more biomarkers to guide treatment of a patient's eye including obtaining one or more initial samples from a biomatrix of the patient prior to an initial treatment on the eye, obtaining one or more first subsequent samples from a biomatrix of the patient after the initial treatment, obtaining one or more second subsequent samples from a biomatrix of the patient after obtaining the first subsequent samples, mixing the initial samples and the first subsequent samples and the second subsequent samples in compositions including one or more protease inhibitors, measuring an initial concentration of one or more biomarkers in the initial samples, monitoring changes in the initial concentration of the one or more biomarkers in the first subsequent samples, and identifying when the concentration of the one or more biomarkers in the second subsequent sample returns to a predetermined threshold nearer the initial concentration. In some embodiments, the predetermined threshold is the concentration of one or more biomarkers in the initial sample. In some embodiments, the biomatrix includes the patient's blood components (e.g., serum, plasma), peripheral blood mononuclear cells, vitreous humor, aqueous humor, or combinations thereof. In some embodiments, the biomarker is measured in one or more of the patient's blood cellular components, serum, plasma, or combinations thereof. In some embodiments, the one or more biomarkers includes levels of one or more peptides, relative levels of the one or more peptides, activity levels of the one or more peptides, relative activity levels of the one or more peptides, expression levels of one or more genes for the one or more peptides, relative expression levels of one or more genes for the one or more peptides, or combinations thereof. In some embodiments, the peptides include MMPs, TIMPs, RAAS components including but not limited to Ang(1-9), AngII, Ang(1-7), Ang(1-10), MasR, AT1R, AT2R, angiotensin converting enzyme-1 (ACE1), ACE2, neprilysin (NEP), aminopeptidase isoforms, or combinations thereof. In some embodiments, obtaining one or more first subsequent samples from a biomatrix of the patient after the initial treatment occurs between about 1 hour and about 4 days after the initial treatment. In some embodiments, the samples are collected in containers including one or more protease inhibitors. In some embodiments, the one or more protease inhibitors includes but is not limited to ethylenediaminetetraacetic acid (EDTA), 1,10-phenanthroline, pepstatin A, angiotensin converting enzyme inhibitors, phenylmethylsulfonyl fluoride (PMSF), or combinations thereof.

Aspects of the present disclosure are directed to a method for guiding treatment of a patient's eye including identifying dry age-related macular degeneration in a patient, obtaining a first biomarker sample from the patient's blood, performing an initial subthreshold laser treatment on the eye, measuring an initial concentration of one or more biomarkers in the first biomarker sample, obtaining a plurality of subsequent biomarker samples from a biomatrix of the patient beginning about 1 hour after the initial subthreshold laser treatment, measuring a concentration of the one or more biomarkers in the subsequent biomarker samples, identifying when the concentration of the one or more biomarkers in the subsequent biomarker samples return to the initial concentration, and performing a subsequent subthreshold laser treatment of the eye. In some embodiments, the one or more biomarkers includes levels of one or more peptides, relative levels of the one or more peptides, activity levels of the one or more peptides, relative activity levels of the one or more peptides, expression levels of one or more genes for the one or more peptides, relative gene expression levels of one or more genes for the one or more peptides, or combinations thereof. In some embodiments, the subthreshold laser is a frequency-double Nd:YLF laser. In some embodiments, the peptides include MMP, TIMPs, RAAS components such as but not limited to Ang(1-9), AngII, Ang(1-7), Ang(1-10), MasR, AT1R, AT2R, ACE1, ACE2, neprilysin (NEP), aminopeptidase isoforms, or combinations thereof.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a chart of a method for identifying and quantifying one or more biomarkers to guide treatment of a patient's eye according to some embodiments of the present disclosure;

FIG. 2 is a chart of a method for guiding treatment of a patient's eye according to some embodiments of the present disclosure;

FIG. 3 is a chart of a method for guiding treatment of a patient's eye according to some embodiments of the present disclosure;

FIGS. 4A-4H portray images of clinical assessments of mouse retina undergoing R:GEN treatment;

FIGS. 5A-5F portray histopathological images of laser-treated retina and Retinal Pigment Epithelial (RPE) cells;

FIGS. 6A-6E portray graphs showing retinal layer expression of renin-angiotensin-aldosterone system (RAAS) components;

FIGS. 7A-7D portray graphs showing RPE layer expression of matrix metalloprotease (MMP) biomarkers;

FIGS. 8A-8E portray graphs showing RPE layer expression of RAAS components;

FIG. 9 is a schematic diagram of the RAAS pathway;

FIGS. 10A-10C portray graphs showing quantification of RAAS peptides using a metabolomic liquid chromatography—mass spectrometry (LC-MS) assay;

FIGS. 11A-11B portray graphs showing unseparated eye cup expression of MMP and tissue inhibitors of metalloprotease (TIMP) biomarkers; and

FIGS. 12A-12C portray graphs showing unseparated eye cup expression of RAAS components.

### DETAILED DESCRIPTION

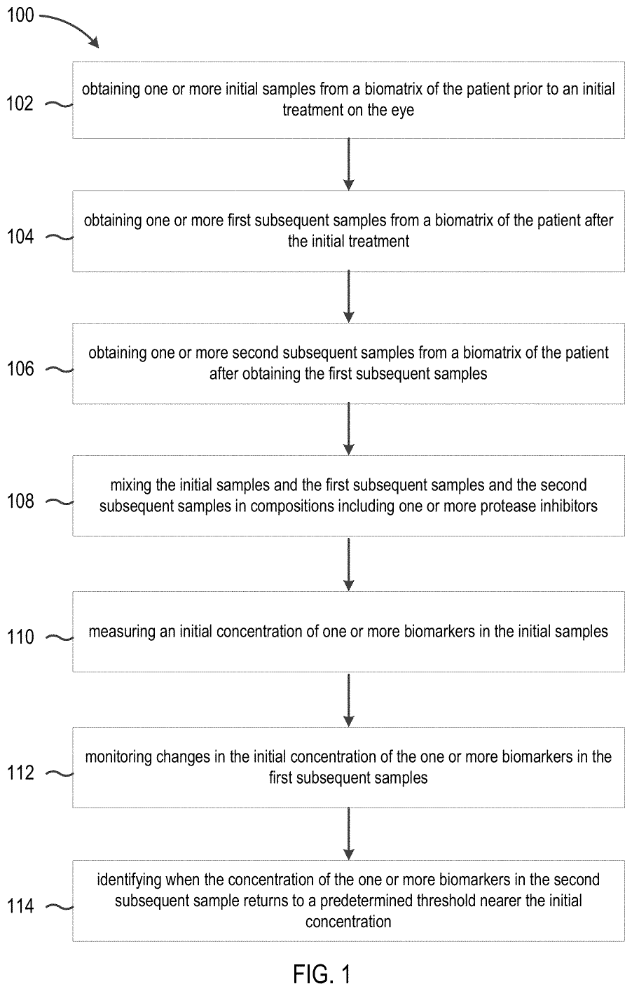

Referring now to FIG. 1, some embodiments of the present disclosure are directed to a method 100 of identifying and quantifying one or more biomarkers to guide treatment in a patient. In some embodiments, the treatment of the patient is of the patient's eye. As used herein, the term “treatment of the eye” includes treatment of or to the eyeball or specific components of the eyeball themselves, as well as associated anatomy such as bone, e.g., the orbit; extraocular muscles, e.g., the superior oblique, the superior rectus, etc.; the eyelid; the conjunctiva; the optic nerve, and the like. In some embodiments, the treatment includes laser stimulation of the eye. In some embodiments, the laser is a subthreshold laser. In some embodiments, the laser is a frequency-double neodymium-doped yttrium lithium fluoride (Nd:YLF) laser. In some embodiments, the treatment includes laser stimulation of the eye in combination with one or more additional therapies.

At 102, one or more initial samples are obtained from a biomatrix of the patient prior to an initial treatment, e.g., on the patient's eye. The biomatrix is a medium present in the patient from which a concentration of a biomarker can be measured. In some embodiments, the biomatrix is a portion of a component of the patient's eye. In some embodiments, the biomatrix includes the patient's blood, peripheral blood mononuclear cells, vitreous humor, aqueous humor, or combinations thereof. In some embodiments, the biomarker is measured in one or more of the patient's blood cellular components, serum, plasma, or combinations thereof. In some embodiments, the biomarker is a molecule, compound, polynucleotide, polypeptide, polysaccharide, etc. having a measurable concentration in the biomatrix prior to the initial treatment, at the time of the initial treatment, subsequent to the initial treatment, or combinations thereof. In some embodiments, the one or more biomarkers includes levels of one or more peptides, relative levels of the one or more peptides, activity levels of the one or more peptides, relative activity levels of the one or more peptides, expression levels of one or more genes for the one or more peptides, relative expression levels of one or more genes for the one or more peptides, or combinations thereof. In an exemplary embodiment, the one or more biomarkers include proteases, e.g., metalloproteases (MMP); tissue inhibitors of metalloproteases (TIMPs); peptides and metabolites associated with the renin-aldosterone angiotensin system (RAAS); receptors and enzymes involved in the RAAS; and the like. Obtaining the one or more initial samples, as well as other samples discussed in the present disclosure, can be obtained via any process suitable to maintain the biomarker disposed therein in a condition to be quantified. In some exemplary examples, the one or more samples can be drawn intravenously, excised from patient tissues, etc.

At 104, one or more first subsequent samples are obtained from a biomatrix of the patient after the initial treatment. At 106, one or more second subsequent samples are obtained from a biomatrix of the patient after obtaining the first subsequent samples. In some embodiments, obtaining 106 one or more second subsequent samples includes obtaining a plurality of samples at regular or irregular intervals. In some embodiments, the samples obtained at 104 and 106 are obtained from the same biomatrix as the initial samples, e.g., each from the patient's blood, eye, etc., from different biomatrices, or combinations thereof. In some embodiments, at 108, the initial samples, first subsequent samples, second subsequent samples, or combinations thereof are mixed in compositions including one or more protease inhibitors. In some embodiments, the samples are collected in containers including one or more protease inhibitors. The protease inhibitors act to prevent the breakdown of peptide and/or peptide activity biomarkers within the samples. In some embodiments, the one or more protease inhibitors include ethylenediaminetetraacetic acid (EDTA), 1,10-phenanthroline, pepstatin A, angiotensin converting enzyme inhibitors, phenylmethylsulfonyl fluoride (PMSF), and the like, or combinations thereof. In some embodiments, the protease inhibitor is provided at a final concentration that is 5.0-100×above 50% inhibitory or effective concentrations (IC₅₀ or EC₅₀). In some embodiments, obtaining 104 one or more first subsequent samples from a biomatrix of the patient after the initial treatment occurs between substantially immediately after the initial treatment to about 5 days after the initial treatment. In some embodiments, obtaining 104 one or more first subsequent samples from a biomatrix of the patient after the initial treatment occurs between about 0.01 hours to about 5 days after the initial treatment. In some embodiments, obtaining 104 one or more first subsequent samples from a biomatrix of the patient after the initial treatment occurs between about 0.05 hours to about 5 days after the initial treatment. In some embodiments, obtaining 104 one or more first subsequent samples from a biomatrix of the patient after the initial treatment occurs between about 0.1 hours to about 5 days after the initial treatment. In some embodiments, obtaining 104 one or more first subsequent samples from a biomatrix of the patient after the initial treatment occurs between about 0.5 hours to about 5 days after the initial treatment. In some embodiments, obtaining 104 one or more first subsequent samples from a biomatrix of the patient after the initial treatment occurs between about 1 hour to about 5 days after the initial treatment. In some embodiments, obtaining 104 one or more first subsequent samples from a biomatrix of the patient after the initial treatment occurs between about 1 hour and about 4 days after the initial treatment. In some embodiments, obtaining 104 one or more first subsequent samples from a biomatrix of the patient after the initial treatment occurs between about 1 day and about 5 days after the initial treatment. In some embodiments, obtaining 104 one or more first subsequent samples from a biomatrix of the patient after the initial treatment occurs between about 1 day and about 4 days after the initial treatment. In some embodiments, obtaining 104 one or more first subsequent samples from a biomatrix of the patient after the initial treatment occurs between about 2 days and about 4 days after the initial treatment. In some embodiments, obtaining 104 one or more first subsequent samples from a biomatrix of the patient occurs after the initial treatment occurs about 3 days after the initial treatment. In some embodiments, obtaining 106 one or more second subsequent samples from a biomatrix of the patient after obtaining the first subsequent samples occurs about 7 days after the initial treatment. In some embodiments, obtaining 106 one or more second subsequent samples from a biomatrix of the patient after obtaining the first subsequent samples occurs more than 7 days after the initial treatment. In some embodiments, obtaining 106 one or more second subsequent samples from a biomatrix of the patient after obtaining the first subsequent samples occurs more than 14 days after the initial treatment. In some embodiments, obtaining 106 one or more second subsequent samples from a biomatrix of the patient after obtaining the first subsequent samples occurs about 1 month after the initial treatment. In some embodiments, obtaining 106 one or more second subsequent samples from a biomatrix of the patient after obtaining the first subsequent samples occurs more than about 1 month after the initial treatment.

At 110, an initial concentration of one or more biomarkers in the initial samples is measured. At 112, the one or more biomarkers in the first subsequent samples are monitored for changes in the initial concentration of those biomarkers. At 114, it is identified when the concentration of the one or more biomarkers in the second subsequent samples return to a predetermined threshold nearer the initial concentration. In some embodiments, biomarkers are quantified within the biomatrix, e.g., in situ, in vitro, or combinations thereof. In some embodiments, the biomarker is concurrently or subsequently separated from a bulk of biomatrix and quantified separately. The biomarkers are measured/quantified by any suitable testing procedure for the specific biomarker, e.g., spectroscopy, chromatography, reverse transcriptase-polymerase chain reaction, etc. In some embodiments, the predetermined threshold is being within about 25% concentration of one or more biomarkers in the initial sample. In some embodiments, the predetermined threshold is being within about 20% concentration of one or more biomarkers in the initial sample. In some embodiments, the predetermined threshold is being within about 15% concentration of one or more biomarkers in the initial sample. In some embodiments, the predetermined threshold is being within about 10% concentration of one or more biomarkers in the initial sample. In some embodiments, the predetermined threshold is being within about 5% concentration of one or more biomarkers in the initial sample. In some embodiments, the predetermined threshold is being within about 1% concentration of one or more biomarkers in the initial sample. In some embodiments, the predetermined threshold is the concentration of one or more biomarkers in the initial sample.

By measuring, quantifying, and monitoring one or more biomarkers in a patient undergoing therapy on of the eye, practitioners are able to monitor the healing progress at the treatment site and better plan subsequent steps in the patient's course of treatment. The biomarker levels identified in the initial sample indicate a baseline or background for the patient, i.e., a level consistent with that which is present when the patient in not generating an active healing response. Treatment of the patient's eye will induce the active healing response, affecting changes in the biomarkers in the biomatrix in the days and weeks that follow. In some embodiments, the concentration or activity of a target biomarker will increase or decrease after treatment before returning to a level substantially similar to baseline. In some embodiments, the concentration or target activity of the target biomarker will initially increase relative to baseline then subsequently decrease relative to baseline, or vice-a-versa, before stabilizing again at a level substantially similar to baseline. Obtaining first subsequent samples at 104 and second subsequent samples at 106 allow a practitioner to monitor the healing response in the patient, with deviations from the baseline indicating the ongoing effects of the healing response. When biomarker levels in the second subsequent samples return to baseline levels resembling those found at step 102, the practitioner can understand that to mean that the patient has recovered from the initial treatment and the condition of the patient's tissue is suitable for subsequent treatment. Thus, some embodiments of the present provide improved information regarding when safe and effective retreatment of a patient's eye using a laser can be commenced.

In one embodiment, a quantitative metabolomic assay specifically quantifying the levels of systemic RAAS peptide is able to determine whether a patient will need further treatment. Unlike present mass spectrometry biomarker assays, this is a targeted approach that is able to measure the levels of the biomarkers. Without wishing to be bound by theory, the levels of the biomarkers will be constant due the presence of protease inhibitor cocktail that is added into collection tubes. In addition, the level of at least one of these RAAS peptides can determine the amount of laser to be administered to the affected patient. In one embodiment uses a quantitative metabolomic assay form whole blood, plasma, serum or other biomatrix that has been collected with the protease inhibitor cocktail which is able to prevent ex vivo metabolism of the RAAS peptides via ACE1 or ACE2 mediated breakdown, even under −80° C. conditions (see Table 1 below).

TABLE 1
Levels of Ang(1-10) from 20 paired human plasma samples collected
in tubes containing either protease inhibitor cocktail (PIC+) versus
no cocktail (PIC−). The concentration of RAAS peptides
were measured with a quantitative metabolomics assay. This
shows that absence of protease inhibitor cocktail allows for
ex vivo metabolism of Ang(1-10), where levels were approximately
50% less than samples collected with PIC.

| Collection Tubes | Ang(1-10) (ng/mL) | PIC+/PIC− Ratio |
|---|---|---|
| PIC+ | 10.80 ± 13.78* | 2.09 ± 1.08 |
| PIC− | 4.19 ± 3.94 | |

*P-value <= 0.01.

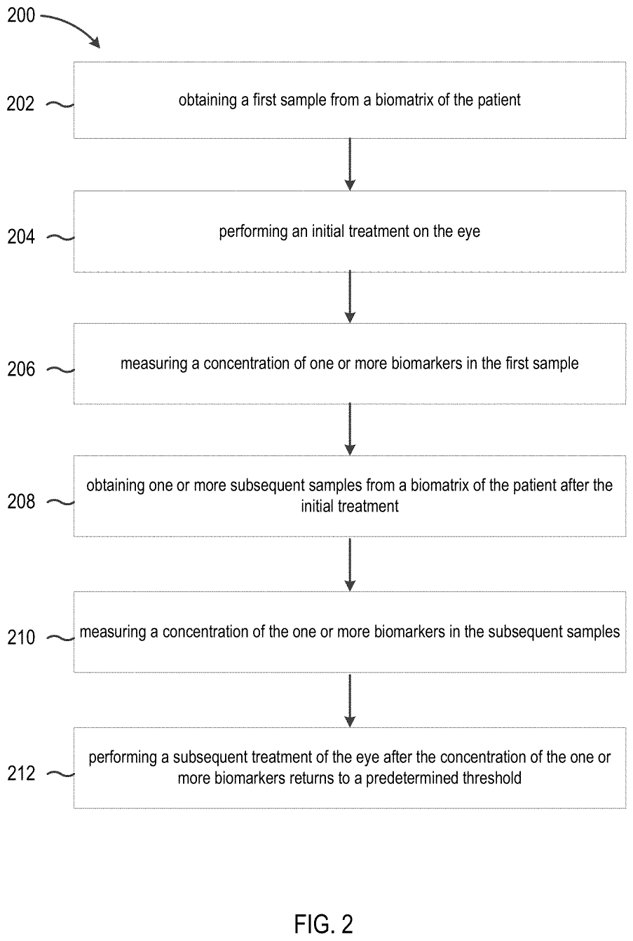

Referring now to FIG. 2, some embodiments of the present disclosure are directed to a method 200 for guiding treatment of a patient's eye. At 202, a first sample is obtained from a biomatrix of the patient. As discussed above, in some embodiments, the biomatrix includes the patient's blood, peripheral blood mononuclear cells, vitreous humor, aqueous humor, or combinations thereof. In some embodiments, the biomarker is measured in one or more of the patient's blood cellular components, serum, plasma, or combinations thereof. As discussed above, in some embodiments, the one or more biomarkers include levels of one or more peptides, relative levels of the one or more peptides, activity levels of the one or more peptides, relative activity levels of the one or more peptides, expression levels of one or more genes for the one or more peptides, relative expression levels of one or more genes for the one or more peptides, or combinations thereof. In some embodiments, the peptides include MMPs, TIMPs, peptides associated with the RAAS, e.g., Ang(1-9), AngII, Ang(1-7), Ang(1-10), MasR, AT1R, AT2R, angiotensin converting enzyme-1 (ACE1), ACE2, neprilysin (NEP), aminopeptidase isoforms, or combinations thereof.

At 204, an initial treatment on the patient's eye is performed. In some embodiments, the initial treatment includes laser stimulation of the eye. In some embodiments, the laser is a subthreshold laser. In some embodiments, the laser is a frequency-double Nd:YLF laser. In some embodiments, the treatment includes stimulation of the eye using a laser in combination with one or more additional therapies.

At 206, a concentration of one or more biomarkers in the first sample is measured in the first sample. As discussed above, this measurement provides a baseline or background level consistent with the patient without an active healing response. At 208, one or more subsequent samples are obtained from a biomatrix of the patient after the initial treatment. In some embodiments, obtaining 208 one or more subsequent samples from a biomatrix of the patient after the initial treatment occurs substantially immediately after the initial treatment. In some embodiments, obtaining 208 one or more subsequent samples from a biomatrix of the patient after the initial treatment occurs at least 0.01 hour after the initial treatment. In some embodiments, obtaining 208 one or more subsequent samples from a biomatrix of the patient after the initial treatment occurs at least 0.05 hour after the initial treatment. In some embodiments, obtaining 208 one or more subsequent samples from a biomatrix of the patient after the initial treatment occurs at least 0.1 hour after the initial treatment. In some embodiments, obtaining 208 one or more subsequent samples from a biomatrix of the patient after the initial treatment occurs at least 1 hour after the initial treatment. In some embodiments, obtaining 208 one or more subsequent samples from a biomatrix of the patient after the initial treatment occurs at least 1 day after the initial treatment. In some embodiments, obtaining 208 one or more subsequent samples from a biomatrix of the patient after the initial treatment occurs at least 2 days after the initial treatment. In some embodiments, obtaining 208 one or more subsequent samples from a biomatrix of the patient after the initial treatment occurs at least 3 days after the initial treatment. In some embodiments, obtaining 208 one or more subsequent samples from a biomatrix of the patient after the initial treatment occurs at least 4 days after the initial treatment. In some embodiments, obtaining 208 one or more subsequent samples from a biomatrix of the patient after the initial treatment occurs between about 1 hour to about 3 days after the initial treatment. As discussed above, in some embodiments, a plurality of subsequent samples are obtained at regular or irregular intervals. At 210, a concentration of the one or more biomarkers is measured in the subsequent samples. At 212, a subsequent treatment of the eye is performed after the concentration of the one or more biomarkers returns to a predetermined threshold. As discussed above, in some embodiments, the predetermined threshold is being within about 25% concentration of one or more biomarkers in the first sample. In some embodiments, the predetermined threshold is being within about 20% concentration of one or more biomarkers in the first sample. In some embodiments, the predetermined threshold is being within about 15% concentration of one or more biomarkers in the first sample. In some embodiments, the predetermined threshold is being within about 10% concentration of one or more biomarkers in the first sample. In some embodiments, the predetermined threshold is being within about 5% concentration of one or more biomarkers in the first sample. In some embodiments, the predetermined threshold is being within about 1% concentration of one or more biomarkers in the first sample. In some embodiments, the predetermined threshold is the concentration of the one or more biomarkers in the first sample. In some embodiments, the subsequent treatment occurs at least 7 days after the initial treatment. In some embodiments, the subsequent treatment occurs at least 14 days after the initial treatment. In some embodiments, the subsequent treatment occurs at least 1 month after the initial treatment. In some embodiments, the subsequent treatment occurs more than 1 month after the initial treatment. In some embodiments, the subsequent treatment includes laser stimulation of the eye.

As a summary of an example of the application of embodiments of the present technology, 42 μJ to 59 μJ were applied around the optic nerve to 12-month-old C57BL/J using R:GEN laser. Clinical assessment, including fundus, optical coherence tomography (OCT), and fluorescein angiography (FA) images were performed at day 0.04, 0.16, 3 and 7. Retina and RPE were histologically assessed using haematoxylin and eosin (H&E) staining and ZO-1 immunostaining. Molecular characterization targeting MMPs and RAAS used reverse transcriptase-polymerase chain reaction (RT-PCR).

Confirmation of the RPE specific changes was verified by FA with no lesion expansion over time. OCT was unable to detect changes. Histopathology assessment showed specific RPE changes using H&E and ZO-1 staining, where recovery was detected by day 3 corresponding with reduction of MMPs and RAAS enzyme and receptor expression. Retinal and RPE layer showed MMP2 gene expression was upregulated corresponding with downregulation of TIMP2 which returned back to baseline values by day 7. RPE gene expression of AT1R and mitochondria assembly protein receptor (MasR), the receptor for Angiotensin (1-7) were statistically increased (p<0.05) at 4 hours after laser application. Additionally, RPE expression of ACE1 and ACE2 were elevated at 1 hour after laser treatment. These ocular findings were detectable using systemic quantitative metabolomic analyses showing Ang (1-9), Ang (1-8) and Ang (1-7) concentrations were increased 1 hour after laser administration.

In this example of an embodiment of the technology, the Q-switched frequency-double Nd:YLF in combination with the RTF technology selectively stimulated RPE. Minor retina changes include activation MMP2 where expression for all MMPs were downregulated by day 3. RPE analyses showed increased RAAS components after treatment which promotes non-fibrotic healing.

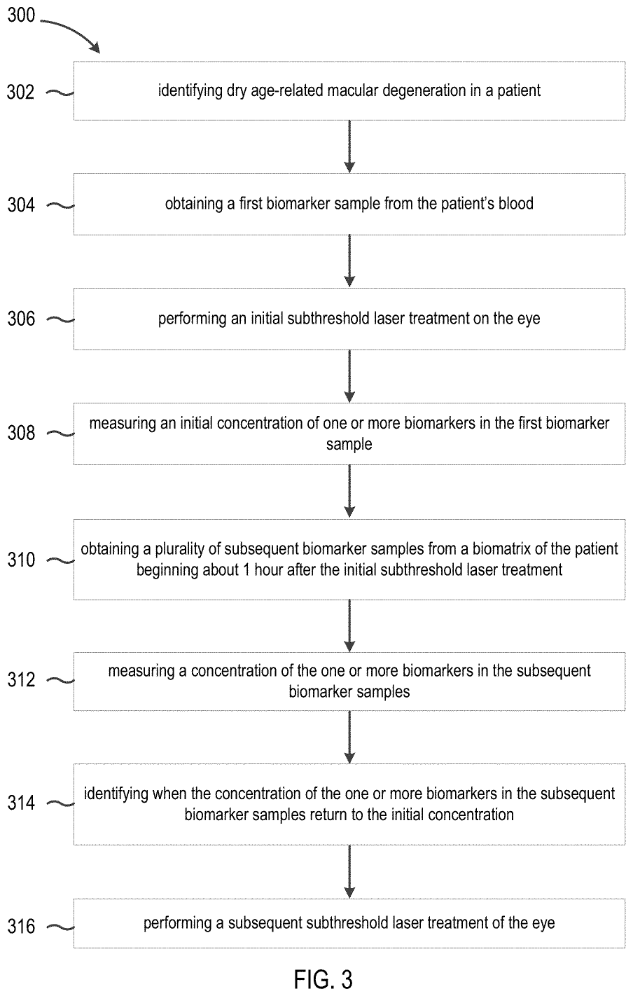

Referring now to FIG. 3, some embodiments of the present disclosure are directed to a method 300 for guiding treatment of a patient's eye. In some embodiments, at 302, dry age-related macular degeneration is identified in a patient. At 304, a first biomarker sample is obtained from the patient's blood. At 306, an initial subthreshold laser treatment is performed on the patient's eye. As discussed above, in some embodiments, the subthreshold laser is a frequency-double Nd:YLF laser. In some embodiments, the treatment includes subthreshold laser treatment in combination with one or more additional therapies. At 308, an initial concentration of one or more biomarkers is measured in the first biomarker sample. As discussed above, in some embodiments, the one or more biomarkers includes levels of one or more peptides, relative levels of the one or more peptides, activity levels of the one or more peptides, relative activity levels of the one or more peptides, expression levels of one or more genes for the one or more peptides, relative expression levels of one or more genes for the one or more peptides, or combinations thereof. In some embodiments, the peptides include MMPs, TIMPs, peptides and components associated with the RAAS, e.g., Ang(1-9), AngII, Ang(1-7), Ang(1-10), MasR, AT1R, AT2R, ACE1, ACE2, neprilysin (NEP), aminopeptidase isoforms, or combinations thereof. At 310, a plurality of subsequent biomarker samples are obtained from a biomatrix of the patient beginning about 2 days after the initial subthreshold laser treatment. At 312, a concentration of the one or more biomarkers is measured in the subsequent biomarker samples. At 314, when the concentration of the one or more biomarkers in the subsequent biomarker samples return to the initial concentration is identified. At 316, a subsequent subthreshold laser treatment of the eye is performed.

### EXAMPLES

Animals. 12-month-old C57BL/J mice were purchased from Charles River where experiments were conducted in accordance with the IACUC Committee approval and guidelines for animal use according to Association for Research and Ophthalmology (ARVO) statement. Mice were anesthetized with ketamine/xylazine mixture before any experimental procedure. Tropicamide eyedrop instillation was followed by pharmacological pupil dilation by 1% tropicamide. Treated animals were divided into four groups, which included 1-, 4-hours, 3- and 7-day time points. Aged-matched healthy untreated animals were included to validate molecular profile and verify clinical changes associated with the laser treatment.

Application of the Selective Retina Therapy. The left eyes were treated using a Q-switched Nd:YLF laser system (R:GEN, Lutronic, Goyang-si, South Korea). In order to establish the threshold energy for the study, a pilot group of C57BL/J mice were treated at different energy and intensities. Once the laser energy was validated by clinical evaluation, fundus pictures, and fluorescein angiography (FA), laser settings were set as laser threshold. Twenty laser spots were applied 360° around the optic nerve. Range energy calculated for each mouse was 42 μJ to 59 μJ, RTF Value of 100% intensity, pulse duration 1.7 μs, spot size of 90 μm and RampAutoStop Mode.

Clinical Assessments. Retinal changes were assessed by three different modalities. By using the Envisu R-Class Bioptigen System (Leyca Microsystems Inc., Buffalo Grove, Ill.) and the rodent posterior pole lens, a full set of pictures from the optic nerve and the retina were acquired at each timepoint. Optical coherence tomography (OCT) sections were collected right after by HRA+OCT Spectralis (Heidelberg Engineering Inc., Franklin, Mass.). Since retinal damage is hard to evaluate once laser is applied, FA was performed to identify retinal leakage. To detect tissue leakage, 10% fluorescein was injected intraperitoneal and the level of leakage was evaluated using both OCT and FA which were acquired simultaneously. OCT scans included a volume scan over leaking areas surrounding optic nerve.

Histopathology. Photoreceptors (PR)s and RPE changes were evaluated by hematoxylin and eosin (H&E) staining. At less three mice per subgroup were included in the analysis. Thus, their eyes were enucleated and submerged into Davidson fixative. Cross-sections were obtained every 5 μm apart, mounted and H&E stained.

Morphological changes in RPE used flatmount immunostained with anti-ZO1 antibodies. Modified flatmount technique was followed based on a previous publication. Anti ZO1 was used as primary antibody (Invitrogen Corporation, Thermo Fisher Scientific, Carlsbad, Calif.) at 1:100 dilution. Images were acquired on Olympus Cellsens Standard Software (Olympus BX-51, Olympus Corporation, Shinjuku-ku, Tokyo).

Gene expression. After ocular enucleation, retina and RPE layers were separated where retina and RPE was homogenized separately using TissueLyser II (QIAGEN, Germantown, Md.) 2 minutes. Total RNA from these tissues was isolated using a 96-well column kit (Quick-RNA 96 kit (Zymo Research, Irvine, Calif.)) and a column kit (RNeasy Micro Kit (QIAGEN, Germantown, Md.)) respectively. Reverse transcription was performed (iScript Reverse Transcription Supermix (Bio-Rad Laboratories, Inc, Hercules, Calif.)). A total of 20 μl from each reaction mix was converted into 50 μl by adding pre-amp supermix (SsoAdvanced PreAmp Supermix, (Bio-Rad Laboratories, Inc, Hercules, Calif.)) and pre amplification assay pool. Ten cycles for preamplification were taken using the Bio-Rad Thermal Cycler (Bio-Rad Laboratories, Inc., Hercules, Calif.). MMPs and RAAS genes (SEQ. ID. NOs. 1-24) were targeted using Biosystems' Quantstudio 12K Flex System (Thermo Fisher Scientific, Carlsbad, Calif.). Cq data was analyzed using DataAssist software version 3.01 (Thermo Fisher Scientific, Carlsbad, Calif.) to obtain fold changes.

Statistical Analysis. Descriptive analysis for the morphological changes, including clinical and histopathology assessments were performed. Gene expression data included means±upper/lower limit from at least 3 independent series of experiments with each experiment done in quadruplicate. When appropriate, data were compared with control using one-way ANOVA or Kruskal Wallis test to determine the significance of our findings. In each of the analysis, p value <0.05 was set as statistical significance. For RT-PCR evaluation, relative changes beyond 0.5 or 2.0-fold difference from control was set as level of statistical significance.

Q-switched Nd:YLF laser treatment. Based on the clinical and histopathological profile obtained throughout the intensity escalation study, laser threshold was validated at 420 to 590. As expected, twenty laser spots were applied surrounding the optic nerve at least 50 μm away from its edge. The average laser energy and target energy used was 56.970 (SD±6.200) and 15.820 (SD±1.790) respectively. An average of 11.99 (SD±3.53) pulses were applied at each spot based on the RTF technology. Thus, laser application was successfully applied onto the mice retina without complication, such as retinal bleeding or breaks. Thus, due to the RTF technology all animals treated were included into the study.

Retinal and RPE changes throughout ancillary testing. Clinical evidence of retinal damage due to the laser treatment assessed by funduscopic images showed minimum pigmentary. Thus, proof of laser spots was guided by fluorescein angiography (FA) on account of retinal changes aforementioned.

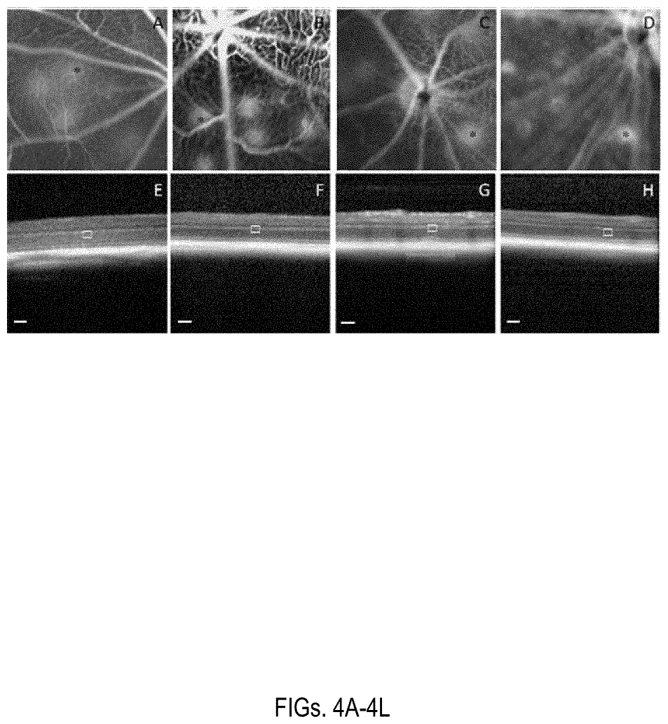

Laser spots were confirmed by fluorescein angiography (FA) at each time point (FIGS. 4A-D). The size of the laser damage did not radially expand over time. OCT imaging was used to visualize changes in the RPE layer. RPE changes were observed as early as four hours after laser administration showed limited distortion in the ellipsoid zone (EZ). Day 3 after laser administration, focal RPE thickening at the laser lesion sites were revealed with elevation of the EZ. By day 7, changes diminished but were still visible (FIGS. 4E-H).

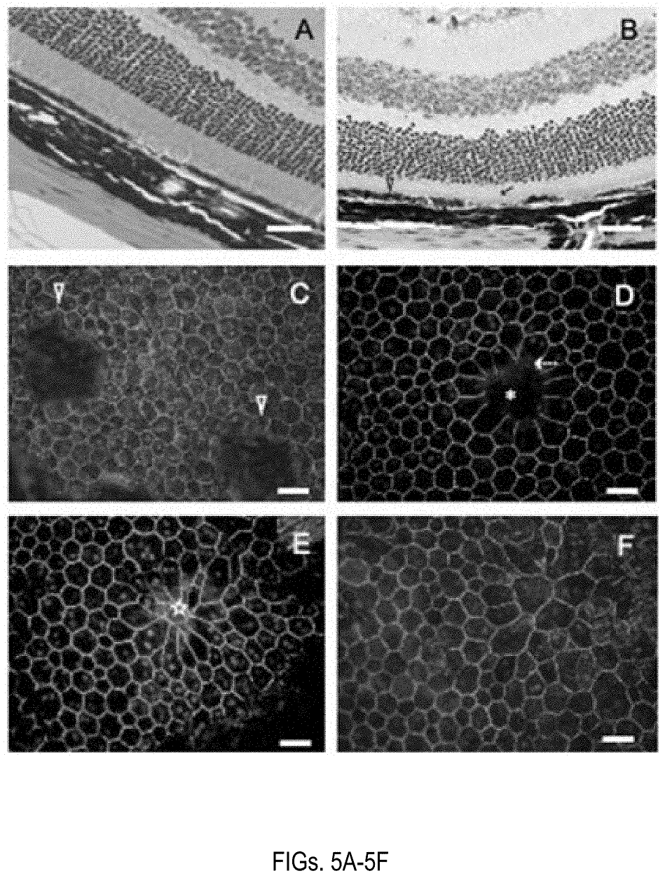

Histopathological response. H&E staining were used to determine histological changes found in the RPE and retina (FIG. 5A-B). Early changes within the RPE included pigmentary disruption (arrows). Disorganization and elongation of the outer segments overlaying the RPE defect was also observed. Progression of the described changes progress over time giving a self-limited spread to the nearby RPE cells within the 100 μm area. Unlike OCT findings, changes within the RPE were wider, such as pigmentary dispersion at the edges of the lesion and cellular lost were observed within the first week after treatment.

RPE flatmount with ZO-1 staining of RPE showed extent of damage over time. Disruption of the tight junctions between contiguous RPE cells was observed (FIG. 5C-F). Immediately after laser application, distinctive disruption of the RPE layer was detected. The approximate size of the lesion was determined to be 100 μm in diameter. At day 3, RPE cell extension has migrated into the laser lesion. By polymegathistic and pleomorphistic characterization, RPE layer was restored back to its cobblestone pattern observed in healthy RPE. Fully enclosed RPE lesions were observed at day 3. However, diffuse staining was observed at that time, which fully disappeared by day 7 given a recovered RPE pattern.

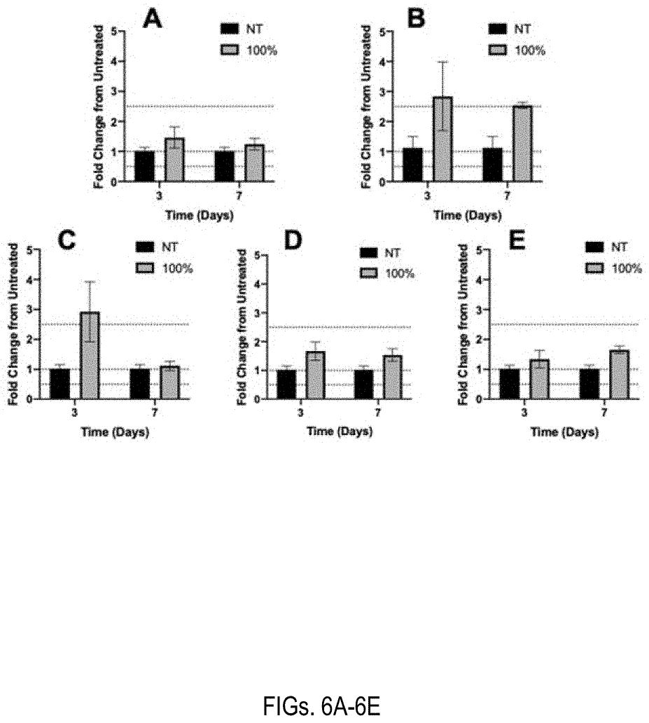

Retinal expression of RAS Components. R:GEN was developed to specifically stimulate melanosome-containing RPE. In FIGS. 6A-6E, the expression of ACE1, ACE2, AT1R, AT2R and MasR in the retina were evaluated. Retinal expression of ACE1 after R:GEN treatment showed no significant changes in the retina expression for these enzymes for both day 3 and day 7. ACE2 expression was significantly increased on day 3, and on day 7 the expression continued to be increased. Retinal layer gene expression for AT1R was increased on day 3 but returned to baseline on day 7. AT1R expression at day 3 was beyond the 2.5-fold threshold for statistical significance. AT2R and MasR gene expression were not statistically different as compared to no treatment for day 3 and 7. These findings suggested that R:GEN laser treatment had minimal effect on retinal gene expression.

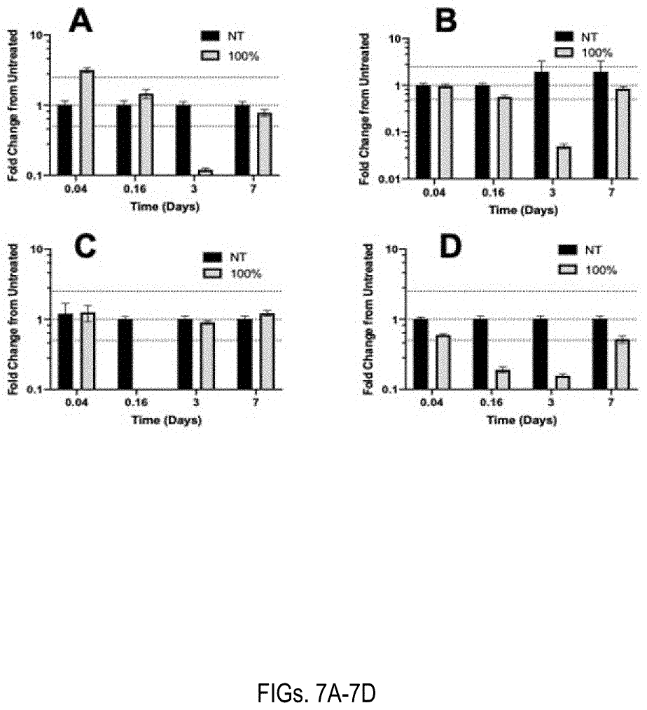

RPE Expression of MMPs. The impact of R:GEN laser treatment on MMP expression found in RPE layer is summarized in FIGS. 7A-7D. The expression of MMP2 was significantly higher at 1 hour (0.04 days) after treatment. Significant reduction in TIMP2 expression began at 4 hours (0.16 days) after treatment, which were statistically reduced. Significant changes in MMP gene expression were detected in RPE layer on day 3, where MMP2, MMP3 and TIMP2 were all significantly reduced, but no changes in MMP9 were detected. These MMPs expression returned to normal levels on day 7.

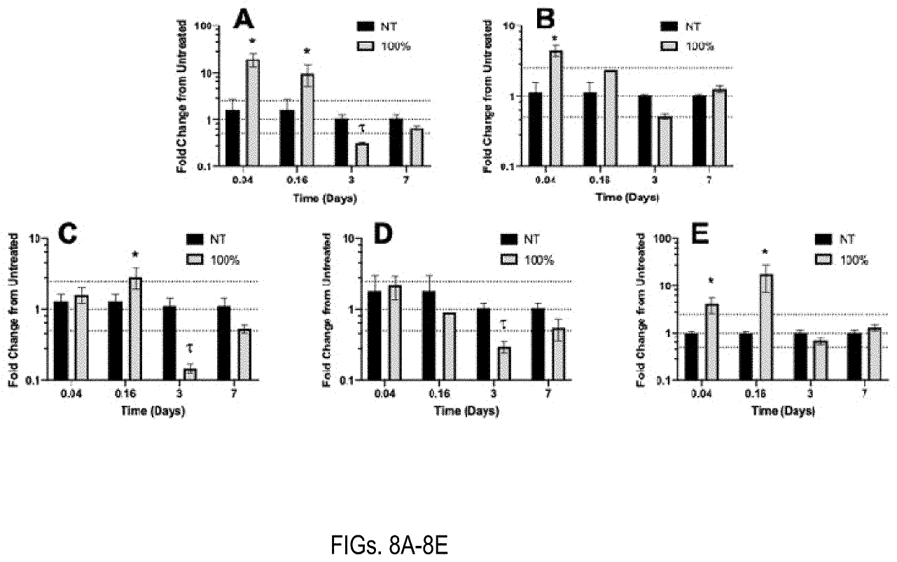

RPE Expression of RAAS Components. Gene expression for RAAS components was also determined in the RPE layer (FIGS. 8A-8E). AT1R and MasR were upregulated 4 hours after treatment (p<0.05). MasR upregulation was detectable as early as 1 hour (0.04 days) and increased its expression at 4 hours. The expression of both AT1R and AT2R in the RPE cells were significantly reduced below the 0.5-fold threshold on Day 3. Similarly, MasR expression was observed elevated at 1 and 4 hours after laser administration (FIG. 8E), which returned to baseline levels on day 3 and 7. These findings corresponded with RPE expression for ACE1 and ACE2 1 hour after laser treatment. While ACE1 continued to significantly elevate at 4 hours after laser treatment, ACE2 expression dropped below statistical significance. Reduction of ACE1 and ACE2 expression reached a nadir on day 3 which returned to baseline levels by day 7.

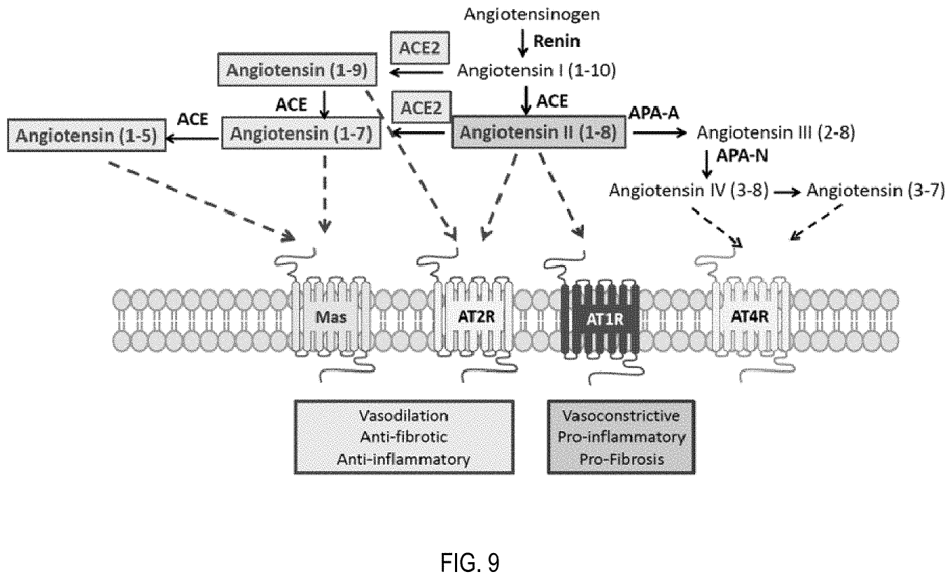

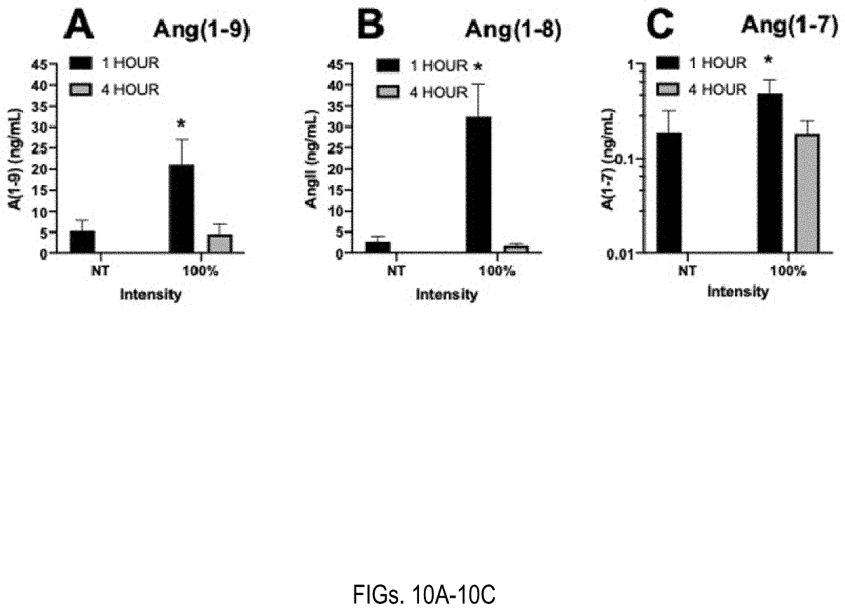

Systemic Metabolomics RAAS. Upregulation of ACE1 and ACE2 expression in RPE layer compelled investigation of the RAAS peptides. Referring now to FIG. 9, as discussed above, the RAAS pathway includes a classic cascade through the AngII/AT1R axis which promotes and accelerate fibrotic healing. Alternative cascades involved in healing and remodeling process include cross-signaling between Ang(1-7)/MasR axis and AngII/Ang(1-9)/AT2R axis which accelerate and promote non-fibrotic healing. Using a metabolomic assay capable of quantifying angiotensin peptides revealed at 1 hour following treatment circulating Ang(1-9), AngII, and Ang(1-7) confirmed the ocular ACE1 and ACE2 expression (FIGS. 10A-10C). Increases in these angiotensin peptides correlated with increased expression of AT1R and MasR expression in the RPE (FIGS. 8C and 8E). Both Ang(1-9) and AngII can bind onto AT2R which can promote non-fibrotic healing. Systemic elevation of Ang(1-7) can traverse through RPE lesions and bind onto MasR which not only promotes non-fibrotic healing but also mobilize progenitors into the injured site.

In one embodiment, the R:GEN laser system developed by Lutronic can preferentially stimulate melanosomes found specifically in RPE. Equipped with OA and RM capable of detecting microbubble formation through changes in light scattering, R:GEN has the feedback mechanism to ensure safety. This was confirmed after a single session of 20 laser spots per treatment, where at the intensity used, R:GEN photomodulation was safe and selectively stimulated RPE.

12-month-old C57BL/J mice (corresponding to humans age 38 to 47 years) evaluated over 1 month did not show irreversible damage in the eye. In this experiment, the response of the retina under different conditions was validated, such as aged cells and level of pigmentation.

Retina leakage was detected immediately after laser application for all intensities, with the escalation of intensity correlated with the level of RPE disruption as shown by H&E. The RPE disruption using ZO-1 flatmount where the lesions showed disruption of the tight junctions was also evaluated. Wound closure over time showed that RPE expanded its boarder using polymegathistic and pleomorphistic mechanisms. In these experiments, the time to lesion healing was on day 3 as shown in the Figures, and targeted RPE cells went through a shorter healing process which was almost completed by day 3 post-treatment instead. Although the gross analysis did not show an increase of the laser spot over the study, the correlation of the laser diameter with the spot size should be a more efficient way to decide if there is any diffusion of the laser.

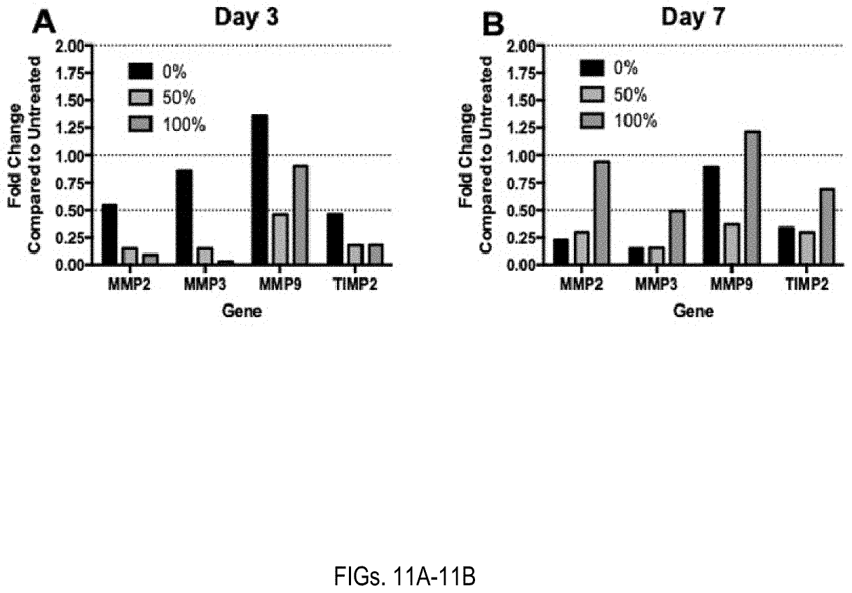

Wound healing by extracellular matrix (ECM) remodeling. MMPs are the important regulator of tissue reparative process and in particular ECM remodeling. MMPs degrade the ECM to regulate intercellular signaling and cell migration. Particularly, MMP2 and MMP9 (both gelatinases), MMP3 (Stromlelysin-1) and TIMP2 are present within healthy RPE cells which degrades ECM components found in the Bruch's membrane. Laser-mediated stimulation of the MMPs has been previously described by others, where a nanosecond laser provided a transient increase of RPE-mediated release of active MMPs. Despite these findings, gene expression changes of MMPs were not detected until day 3. There is consistency across separate studies where, in one study, the entire eye cup was evaluated (see FIGS. 11A-11B), where an intensity dependent reduction of MMP2, -3, and TIMP2 was seen. On day 7, an intensity dependent recovery in gene expression was apparent. In addition, the RPE layer was separated to see whether selective stimulation of the RPE may activate MMPs. MMP2 expression was elevated and non-significant downregulation of TIMP-2 was seen at 1 hour. Similar to the eyecup finding, on day 3, treated RPE were found to have reduction of MMP2, -3, and TIMP2, with full recovery by day 7. MMPs seem to not change in earlier timepoints, where at near lesion resolution, their expressions correspond to RPE healing. Unlike other studies showing changes in MMP9, which was associated with chronic wound healing, in this study MMP9 was not significantly altered. Additionally, MMP2 and TIMP2 expression has been correlated to inflammation and scar formation, however, in the experiments discussed herein, MMP2 was elevated at 1 hour after treatment where its expression along with TIMP2 steadily declined till day 3 and returned to non-treated levels. These findings suggest that R:GEN laser treatment is less likely to induce chronic inflammation.

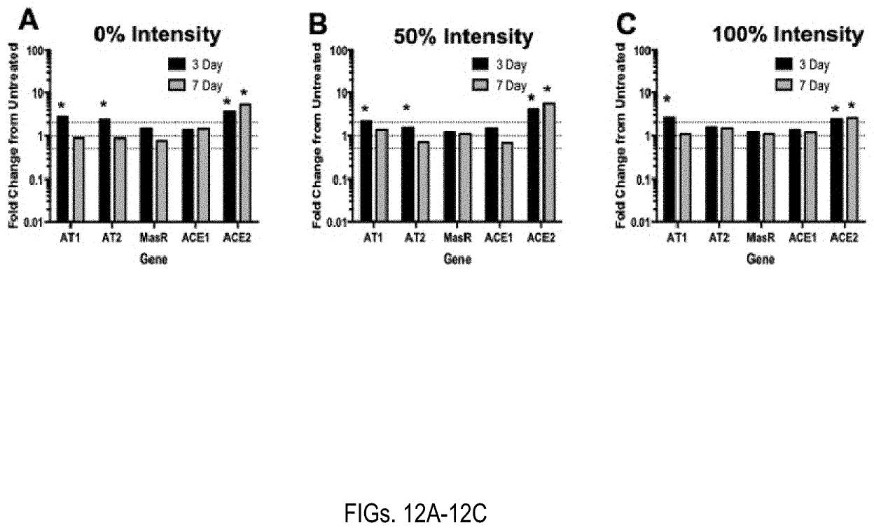

RAAS is an underappreciated pathway associated with tissue repair. Gene expression using entire eyecup (see FIGS. 12A-12C) showed AT2R and MasR were significantly changed over the entire laser intensities spectrum. AT1R expression was elevated on day 3 but returned to baseline by day 7. The expression of ACE1 and ACE2 were significantly elevated at both day 3 and 7 for all three intensities. A difference of gene expression between the photoreceptor and RPE layers was investigated. Retinal layer AT1R expression was statistically above the 2.5-fold threshold at Day 3, and otherwise were consistent with studies using entire eyecup analyses, where no changes in retinal AT2R and MasR expression were seen.

Similar analyses were evaluated using the RPE layer where increased AT1R expression was observed at 4 hours followed by dramatic gene expression downregulation by Day 3 and a rebound to baseline levels by day 7. In contrast, no increased expression of AT2R was detected, but a decline was seen on day 3 corresponding to near complete RPE lesion closure. Interestingly, the expression Mas in RPE showed a dramatic increase, which was not detected in whole eyecup or retinal layer gene expression. To further determine the impact of R:GEN stimulation on the RPE, ACE1, and ACE2 expression in the RPE layer was determined. At 1 hour, greater than 10-fold elevation of ACE1 gene expression was detected which continued at 4 hours. Like other markers of wound healing, a significant reduction was detected on day 3 which returned to baseline by day 7. Similarly, the expression of ACE2 was also detected at 1 hour, with a nadir on day 3 and return to baseline by day 7.

The RAAS peptide levels were quantified using a metabolomic approach, where blood from animals treated with R:GEN laser was collected and compared to untreated mice. One hour after treatment, the levels of Ang(1-9), AngII and Ang(1-7) were all evaluated and concurred with the increase RPE expression of ACE1 and ACE2. The systemic elevation of these peptides suggests that there is not only a local response but also a systemic response after laser induction.

More importantly, RAAS peptide increased expression showed that there is rapid tissue repair mechanism that is activated. AngII binding onto AT1R (AngII/AT1R axis) is thought to be part of the classical RAAS pathway where tissue repair is correlated with fibrosis. AngII has been described to promote healing, where its binding onto AT1R can promote inflammation. In contrast, systemic Ang(1-7) elevation and corresponding RPE MasR expression suggesting a balance with anti-fibrotic wound healing is activated. Ang(1-7)/MasR axis is recognized as a modulator of chronic inflammation and pro-resolving mechanisms. These findings were observed throughout clinical assessments, where OCT and histopathology did not show changes in the reflectivity or fibrotic tissue respectively.

Methods and systems of the present disclosure are advantageous in that they enable more efficient scheduling of therapies in patients undergoing treatments such as laser treatment of the eye. Certain biomarkers, namely MMPs, TIMPs, and compounds associated with RAAS, are effective indicators of the levels of healing response generated by the patients undergoing the therapy. By monitoring the presence and amount of one or more of the above biomarkers, it is determined when the patient's body has sufficiently responded to the previous treatment, such that retreatment may be appropriate. The methods and systems of the present disclosure are effective in the treatment of eye diseases, particularly those, e.g., dry age-related macular degeneration, which utilize laser treatment alone or in combination with other treatments.

Thus, SRT using R:GEN Q-switched frequency-double Nd:YLF laser can be used to selectively stimulate RPE. Minimal molecular changes in the retinal were observed with regards to the RAAS. As previously described, MMP factors were modified within the RPE but were downregulated on day 3 which corresponded to the RPE recovery. Molecular RPE analyses showed that R:GEN stimulation can activate RAAS pathway specifically to prevent ocular fibrosis which is mediated by the expression and elaboration of TGF-β. This data suggests that that R:GEN laser is able to promote non-fibrotic healing.

Studies have demonstrated crosstalk between MMPs and RAAS. In atherosclerosis, MMPs can destabilize plaques through AngII induction which is in the cardiovascular arena. MMP9/TIMP1 imbalance activated Ang(1-7) and ACE expression in atherosclerotic plaques. However there has been no data supporting the role of RAAS in the ocular tissue after the laser treatment or the role of RAAS activation following laser induced RPE wound healing. Some embodiments of the present technology have shown similarities where MMP9 and ACE expression were both elevated after treatment. As discussed above, the RAAS has been shown to be important in managing blood pressure and hemodynamics. These embodiments demonstrate correlation between these two pathways, specifically the RPE cells found in the retina.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A method for guiding treatment of a patient's eye, comprising:
obtaining a first sample from a biomatrix from the patient;
performing an initial treatment on the eye;
measuring a concentration of one or more biomarkers in the first sample;
obtaining one or more subsequent samples from a biomatrix of the patient after the initial treatment;
measuring a concentration of the one or more biomarkers in the subsequent samples; and
performing a subsequent treatment of the eye after the concentration of the one or more biomarkers returns to a predetermined threshold,
wherein the one or more biomarkers include levels of one or more peptides, relative levels of the one or more peptides, activity levels of the one or more peptides, relative activity levels of the one or more peptides, gene expression levels of one or more genes for the one or more peptides, relative expression levels of one or more genes for the one or more peptides, or combinations thereof,
wherein the peptides include matrix metalloproteases (MMP), tissue inhibitors of metalloproteases (TIMPs), RAAS components including, but not limited to, Ang(1-9), AngII, Ang(1-7), Ang(1-10), MasR, AT1R, AT2R, angiotensin converting enzyme-1 (ACE1), ACE2, neprilysin (NEP), aminopeptidase isoforms, or combinations thereof.

2. The method according to claim 1, wherein the predetermined threshold is the concentration of the one or more biomarkers in the first sample.

3. The method according to claim 1, wherein the biomatrix includes the patient's blood, peripheral blood mononuclear cells, vitreous humor, aqueous humor, or combinations thereof.

4. The method according to claim 3, wherein the biomarker is measured in one or more of the patient's blood cellular components, serum, plasma, or combinations thereof.

5. The method according to claim 1, wherein the initial treatment and the subsequent treatment includes laser stimulation of the eye.

6. The method according to claim 5, wherein the laser is a subthreshold laser.

7. The method according to claim 6, wherein the laser is a frequency-double neodymium-doped yttrium lithium fluoride (Nd:YLF) laser.

8. The method according to claim 1, wherein obtaining one or more subsequent samples from a biomatrix of the patient after the initial treatment occurs at least about one hour after the initial treatment.

9. The method according to claim 8, wherein the subsequent treatment occurs at least 7 days after the initial treatment.

10. A method of identifying and quantifying one or more biomarkers to guide treatment of a patient's eye, comprising:
obtaining one or more initial samples from a biomatrix of the patient prior to an initial treatment on the eye;
obtaining one or more first subsequent samples from a biomatrix of the patient after the initial treatment;
obtaining one or more second subsequent samples from a biomatrix of the patient after obtaining the first subsequent samples;
mixing the initial samples and the first subsequent samples and the second subsequent samples in compositions including one or more protease inhibitors;
measuring an initial concentration of one or more biomarkers in the initial samples;
monitoring changes in the initial concentration of the one or more biomarkers in the first subsequent samples; and
identifying when the concentration of the one or more biomarkers in the second subsequent sample returns to a predetermined threshold nearer the initial concentration.

11. The method according to claim 10, wherein the predetermined threshold is the concentration of one or more biomarkers in the initial sample.

12. The method according to claim 10, wherein the biomatrix includes the patient's blood, peripheral blood mononuclear cells, vitreous humor, aqueous humor, or combinations thereof.

13. The method according to claim 12, wherein the biomarker is measured in one or more of the patient's blood cellular components, serum, plasma, or combinations thereof.

14. The method according to claim 10, wherein the one or more biomarkers includes levels of one or more peptides, relative levels of the one or more peptides, activity levels of the one or more peptides, relative activity levels of the one or more peptides, expression levels of one or more genes for the one or more peptides, relative expression levels of one or more genes for the one or more peptides, or combinations thereof.

15. The method according to claim 14, wherein the peptides include matrix metalloproteases (MMP), tissue inhibitors of metalloproteases (TIMPs), RAAS components including Ang(1-9), AngII, Ang(1-7), Ang(1-10), MasR, AT1R, AT2R, angiotensin converting enzyme-1 (ACE1), ACE2, neprilysin (NEP), aminopeptidase isoforms, or combinations thereof.

16. The method according to claim 10, wherein obtaining one or more first subsequent samples from a biomatrix of the patient after the initial treatment occurs between 1 hour and about 4 days after the initial treatment.

17. The method according to claim 10, wherein the samples are collected in containers including one or more protease inhibitors, wherein the one or more protease inhibitors includes ethylenediaminetetraacetic acid (EDTA), 1,10-phenanthroline, pepstatin A, angiotensin converting enzyme inhibitors, phenylmethylsulfonyl fluoride (PMSF), or combinations thereof.

18. A method for guiding treatment of a patient's eye, comprising:
identifying dry age-related macular degeneration in a patient;
obtaining a first biomarker sample from the patient's blood;
performing an initial subthreshold laser treatment on the eye;
measuring an initial concentration of one or more biomarkers in the first biomarker sample;
obtaining a plurality of subsequent biomarker samples from a biomatrix of the patient beginning about 1 hour after the initial subthreshold laser treatment;
measuring a concentration of the one or more biomarkers in the subsequent biomarker samples;
identifying when the concentration of the one or more biomarkers in the subsequent biomarker samples return to the initial concentration; and
performing a subsequent subthreshold laser treatment of the eye,
wherein the one or more biomarkers includes levels of one or more peptides, relative levels of the one or more peptides, activity levels of the one or more peptides, relative activity levels of the one or more peptides, expression levels of one or more genes for the one or more peptides, relative expression levels of one or more genes for the one or more peptides, or combinations thereof.

19. The method according to claim 18, wherein the subthreshold laser is a frequency-double neodymium-doped yttrium lithium fluoride (Nd:YLF) laser.

20. The method according to claim 18, wherein the peptides include matrix metalloproteases (MMP), tissue inhibitors of metalloproteases (TIMPs), and RAAS components including Ang(1-9), AngII, Ang(1-7), Ang(1-10), MasR, AT1R, AT2R, angiotensin converting enzyme-1 (ACE1), ACE2, neprilysin (NEP), aminopeptidase isoforms, or combinations thereof.
